(19) United States
(12) Patent Application Publication
(10) Pub. No.: US 2019/0167976 A1
(43) Pub. Date: Jun. 6, 2019

(54) CONDUCTIVE HUMAN INTERFACE WITH POLYMERIC ELECTRICAL CONTACT ELEMENT

(71) Applicants: The Ohio Willow Wood Company, Mount Sterling State, OH (US); Infinite Biomedical Technologies, LLC, Baltimore, MD (US)

(72) Inventors: Stephen Byers, Dublin, OH (US); Daniel J. Conway, Columbus, OH (US); Rahul Reddy Kaliki, Baltimore, MD (US); Damini Agarwal, Baltimore, MD (US)

(21) Appl. No.: 16/209,296

(22) Filed: Dec. 4, 2018

Related U.S. Application Data
(60) Provisional application No. 62/594,661, filed on Dec. 5, 2017.

(51) Int. Cl. A61N 1/04; A61F 2/72; A61F 2/78; A61L 31/14

(52) U.S. Cl. CPC A61N 1/0496; A61F 2/72; A61B 2562/14; A61L 31/14; A61B 2562/227; A61F 2/7812

(57) ABSTRACT

An apparatus is provided for conducting electrical signals at the skin of a user. In a given embodiment, the apparatus includes a fabric layer, an electrically insulating coating, and an electrical contact element. The fabric layer has an interior surface and an exterior surface. The coating has an exterior surface overlying the interior surface of the fabric layer, and has an interior surface exposed for contact with the skin of the user. The contact element includes a body of polymeric material with an electrically conductive additive dispersed in the polymeric material. The body of polymeric material reaches through the fabric layer and the coating, has an interior electrical contact surface exposed at the interior surface of the coating, and further has an exterior electrical contact surface exposed at the exterior surface of the fabric layer.

### TECHNICAL FIELD

This technology relates to an electrically conductive human interface for communicating signals between an assistive device and the skin of a user.

### BACKGROUND

An assistive device may function as a supplement to the body of a user. Examples include prosthetic devices, orthotic devices, exoskeletal devices, wheelchairs, and the like. Such an assistive device cooperates with the neuromuscular and skeletal systems of the user to operate under input from the user and/or to provide feedback to the user. This requires the communication of electrical signals such as, for example, transcutaneous electrical nerve stimulation (TENS) signals and electromyographic (EMG) signals, between the user and the assistive device. An electrically conductive human interface is thus provided to cooperate with the assistive device by conducting the electrical signals at the skin of the user.

### SUMMARY

An apparatus is provided for conducting electrical signals at the skin of a user. In a given embodiment, the apparatus includes a fabric layer, an electrically insulating coating, and an electrical contact element. The fabric layer has an interior surface and an exterior surface. The coating has an exterior surface overlying the interior surface of the fabric layer, and has an interior surface exposed for contact with the skin of the user. The contact element includes a body of polymeric material with an electrically conductive additive dispersed in the polymeric material. The body of polymeric material reaches through the fabric layer and the coating, has an interior electrical contact surface exposed at the interior surface of the coating, and further has an exterior electrical contact surface exposed at the exterior surface of the fabric layer.

In another embodiment, the apparatus includes a plurality of electrical contact elements, each of which comprises a body of polymeric material with an electrically conductive additive dispersed in the polymeric material. Each body of polymeric material reaches through the fabric layer and the coating, has an interior electrical contact surface exposed at the interior surface of the coating, with an exterior electrical contact surface exposed at the exterior surface of the fabric layer. The apparatus further includes a separate component including an electrode. The separate component, which can be prosthetic socket, is configured to interconnect the electrode with an assistive device that is operable in electrical communication with the electrode. The separate component is further configured to be placed over the exterior surface of the fabric layer in a predetermined operative position relative to the fabric layer. The electrode is configured to contact one or more of the exterior electrical contact surfaces on the contact elements when the separate component is in the predetermined operative position.

An electrically insulating support structure may be included to support the contact elements in a fixed array separately from the fabric layer and the coating. The support structure can be received between the fabric layer and the coating, embedded fully within the coating, or mounted on the exterior surface of the fabric layer. The support structure may alternatively be configured to be donned as a band encircling a residual limb, or as sleeve with an open proximal end and a closed distal end.

### BRIEF DESCRIPTION OF THE DRAWINGS

FIG. 1 is a front sectional view of a prosthetic liner equipped with electrical contact elements.

FIG. 2 is an enlarged view of parts shown in FIG. 1.

FIG. 3 is a front view, partly in section, of a socket for use with the liner of FIG. 1.

FIG. 4-10 are sectional view similar to FIG. 2, each of which shows a respective alternative embodiment of an electrical contact element.

FIG. 11 is a perspective view of a support structure with electrical contact elements mounted on the support structure.

FIG. 12 is a sectional view of parts shown in FIG. 11.

FIG. 13 is a view similar to FIG. 12, showing an additional part of an apparatus that includes the support structure of FIG. 11.

FIGS. 14 and 15 are views similar to FIG. 12, showing parts in alternative arrangements.

FIG. 16 is a perspective view of an electrode device.

FIG. 17 is a side view of the electrode device of FIG. 16.

FIG. 18 is a perspective view of an alternative support structure for electrical contact elements.

FIG. 19 is a front view of an alternative support structure for electrical contact elements.

FIG. 20 is a view similar to FIG. 1 showing an alternative embodiment of the apparatus.

FIG. 21 is an enlarged view of parts shown in FIG. 20.

### DETAILED DESCRIPTION

The embodiments illustrated in the drawings have parts that are examples of the elements recited in the claims. The illustrated embodiments thus include examples of how a person of ordinary skill in the art can make and use the claimed invention. They are described here to meet the enablement and best mode requirements of the patent statute without imposing limitations that are not recited in the claims. One or more of the elements of one embodiment may be used in combination with, or as a substitute for, one or more elements of another as needed for any particular implementation of the claimed invention.

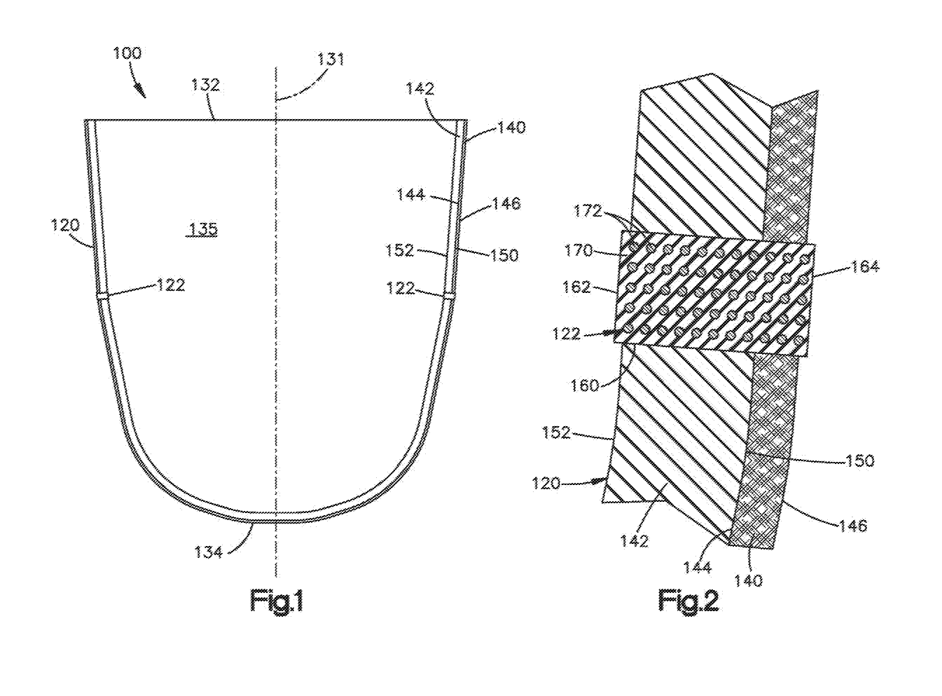

The apparatus 100 shown in FIG. 1 includes a liner 120 for a prosthetic socket. The liner is receivable over a user's residual limb. The apparatus 100 further includes electrical contact elements 122 mounted on the liner 120. The contact elements 122 are configured to communicate electrical signals with the skin of the user at the residual limb and, together with the liner 120, to provide an interface between the user and an assistive device (not shown). The assistive device can be any device that cooperates with the neuromuscular and skeletal system of the user. Such devices include, for example, prosthetic devices, orthotic devices, exoskeletal devices, powered wheelchairs, and the like. While the example of FIG. 1 relates to a prosthetic liner 120, the apparatus 100 can alternatively include a sleeve, a band, a pad, or any other suitable device configured for contact with the skin of the user. In each case, any suitable number of the contact elements 122 can be provided as needed for communicating the assistive device with the appropriate neuromuscular structure of the user.

The liner 120 has a generally conical shape with a longitudinal central axis 131, an open proximal end 132 through which the residual limb projects into the liner 120, and a closed distal end 134. In the illustrated example, the liner 120 includes a fabric layer 140 and a soft coating 142. The fabric layer 140 has an interior surface 144 covered by the soft coating 142. The fabric layer 140 further has an exterior surface 146 which, as shown in FIG. 1, is exposed as the outermost surface of the liner 120. The soft coating 142 has an exterior surface 150 overlying the interior surface 144 of the fabric layer 140. The soft coating 142 further has an interior surface 152 exposed for overlying contact with the user's skin at the residual limb.

The fabric layer 140 is configured to form a flexible substrate. Suitable materials include, for example, stretch controlling fabrics, stretchable non-woven materials, fiber-on-end fabrics, and the like. A stretch-controlling fabric can be more stretchable in one direction than another direction. For example, a stretch-controlling fabric can have a limited stretch direction that is substantially orthogonal to a non-limited stretch direction. In the example shown in FIG. 1, the stretch-controlling fabric is oriented to permit greater stretch in a radial or circumferential direction than in an axial or other longitudinal direction.

The soft coating 142 is provided for comfortable long term wear. As shown in FIG. 2, the soft coating 142 is an electrically insulating body of elastomeric material. As used herein, the term “insulating” means that the material can be classified as an electrical insulator, i.e., a material having sufficiently high resistivity to substantially prevent current flow when exposed to operating voltages of the device. The elastomeric material may comprise a soft polymer such as, for example, thermoplastic elastomers (TPE), silicones, block copolymers, urethanes, or the like.

Each contact element 122 preferably has the configuration of the contact element 122 shown for example in FIG. 2. Each contact element 122 thus has a peripheral side surface 160 reaching fully between first and second opposite end surfaces 162 and 164. The contact element 122 reaches through the fabric layer 140 and the soft coating 142 such that the first end surface 162 is exposed at the interior surface 152 of the soft coating 142 and the second end surface 164 is exposed at the exterior surface 146 of the fabric layer 140. The contact element 120 may protrude slightly beyond the interior and exterior surfaces 152 and 146, with the first and second end surfaces 162 and 164 raised slightly from those surfaces 152 and 146, as shown. In this arrangement the first end surface 162 serves as an interior electrical contact surface exposed for contact with the skin of the user to receive EMG signals produced by muscles of the user, or to transmit TENS or other electrical signals to the skin of the user. The second end surface 164 serves as an exterior electrical contact surface exposed for contact with an electrode at a separate component, such as a socket in which the liner 120 is received, as described below.

As shown schematically in FIG. 2, each contact element 122 is formed as a body of polymeric material 170 in which an electrically conductive additive is dispersed to impart electrical conductivity. The polymeric material can include TPE, silicones, block copolymers, urethanes, or the like, and may have any one or more of the specific compositions and properties disclosed in copending U.S. patent application Ser. No. 15/726,624, filed Oct. 6, 2017, which is incorporated by reference in its entirety. For example, the polymeric material 170 may comprise a medical grade silicone such as Dragon Skin® 30 silicone by Smooth-On, Inc. of Macungie, Pa., USA.

The electrically conductive additive may comprise conductive particles and/or strands of materials such as, for example, gold, copper, nickel, iron, iron-oxide, silver, carbon, carbon black, carbon nanotubes, graphite, or combinations thereof, and may also may have any one or more of the specific compositions, concentrations, configurations and properties disclosed in the above-noted U.S. patent application Ser. No. 15/726,624 which is incorporated reference. Accordingly, the electrically conductive additive can be provided in the form of conductive particles 172 as shown schematically in FIG. 2.

A socket 200 for use with the liner 120 is shown schematically in FIG. 3. Like the liner 120, the socket 200 has a generally conical shape with a longitudinal central axis 201, a proximal end 204, and a distal end 206. The proximal end 204 is open for receiving the liner 120 with the residual limb. The distal end 206 is closed by an electrical connector 210 with a processing device 212 (shown schematically).

An interior surface 220 of the socket 200 is configured for overlying contact with the exterior surface 146 of the liner 120 when the liner 120 is located within the socket 200. Electrodes 230, which are preferably formed of metal, are mounted on the interior surface 220 of the socket 200. The electrodes 230 are provided to make electrical signal-transmitting contact with the contact elements 122 on the liner 120. Specifically, each electrode 230 is configured and arranged to adjoin the exterior electrical contact surface 164 on one or more of the contact elements 122 when the liner 120 is located in a predetermined operative position in the socket 200. Conductive signal lines 232 communicate the electrodes 230 with the processing device 212 at the connector 210. The contact elements 122 on the liner 120 are thereby connected in signal-transmitting communication with the processing device 212 at the connector 210 when the liner 120 is in the predetermined operative position.

The connector 210 is configured to electrically communicate the processing device 212 with the assistive device. The processing device 212 is configured to transform EMG signals at the contact elements 122 into control signals for the assistive device. Additionally or alternatively, the processing device 212 is configured to transmit electrical signals to the contact elements 122 for TENS, to create information flowing into the body, or to provide feedback from the assistive device.

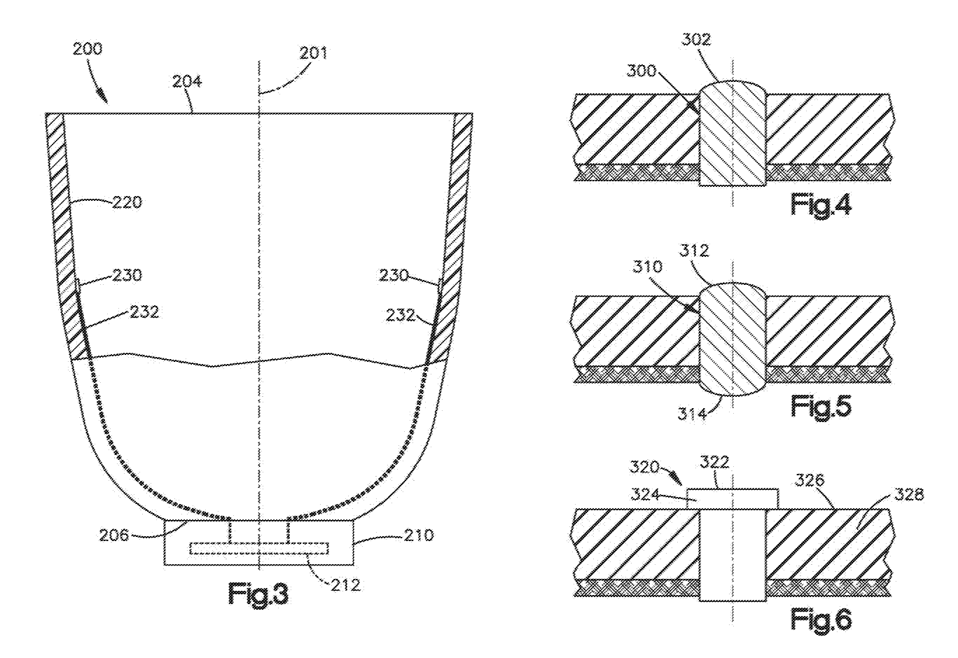

In the embodiment shown in FIG. 2, the peripheral side surface 160 of the contact element 120 is cylindrical with a uniform diameter between planar opposite end surfaces 162 and 164. Alternative embodiments are shown in FIGS. 4-10.

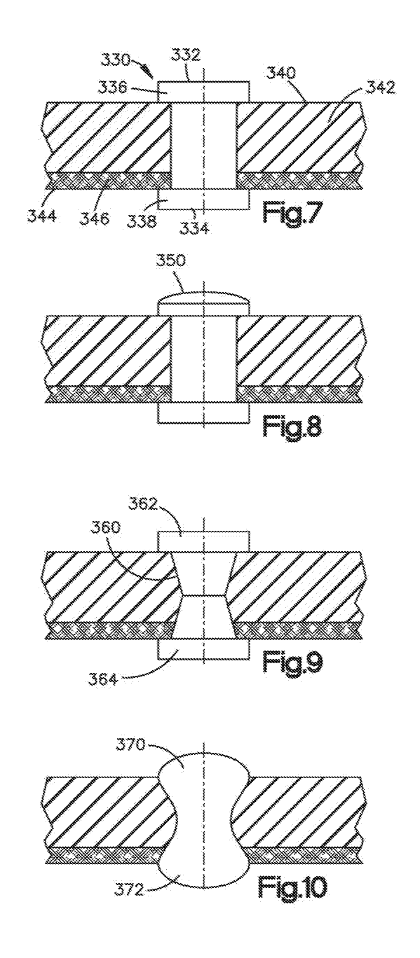

In the alternative of FIG. 4, a contact element 300 has an interior electrical contact surface 303 with a dome-shaped contour. In FIG. 5, a contact element 310 has a dome-shaped contour at both the interior and exterior electrical contact surface 312 and 314. In FIG. 6, a contact element 320 has a planar interior electrical contact surface 322 on an inner end portion 324 that protrudes from, and projects over, the interior surface 326 of the corresponding soft coating 328. In FIG. 7, a contact element 330 has planar interior and exterior electrical contact surfaces 332 and 334 on protruding opposite end portions 336 and 338 that project over the interior surface 340 of the soft coating 342 and the exterior surface 344 of the liner 346, respectively. The embodiment of FIG. 8 differs from that of FIG. 7 where an interior electrical contact surface 350 has a dome-shaped contour. The embodiment of FIG. 9 differs from that of FIG. 7 where a length section 360 between the opposite end portions 362 and 364 is tapered radially inward from the opposite end portions 362 and 364. The embodiment of FIG. 10 has a similarly tapered hourglass configuration between opposite end portions 370 and 372 of an entirely cornerless contact element 374. In each embodiment, the contact element fits closely through the soft coating with the non-conducting material of the contact element adjoining, and is preferably bonded to, the surrounding non-conducting material of the soft coating.

As shown in FIGS. 11 and 12, a support structure 400 also can be provided. The support structure 400 supports a plurality of contact elements 402, each of which is configured as described above. Specifically, the support structure 400 supports the contact elements 402 in a fixed array that is predetermined with reference to multiple electrodes. The array includes distinct groups of contact elements 402 that are arranged for each electrode to make signal-transmitting contact with only one respective group of contact elements 402 without making signal-transmitting contact with any contact element 402 in any other group.

In the illustrated embodiment, the support structure 400 is shaped as a sheet with a uniform thickness between planar opposite side surfaces 412 and 414. The sheet 400 is formed of electrically non-conductive material such as silicone. As shown in enlarged detail FIG. 12, the contact elements 402 are received closely through apertures 415 in the sheet 410 such that the electrical contact surfaces 420 and 422 are equally spaced inward and outward from the corresponding side surfaces 412 and 414. The contact elements 402 can be press-fitted into the apertures 15, but are preferably bonded to the surrounding material of the sheet 400.

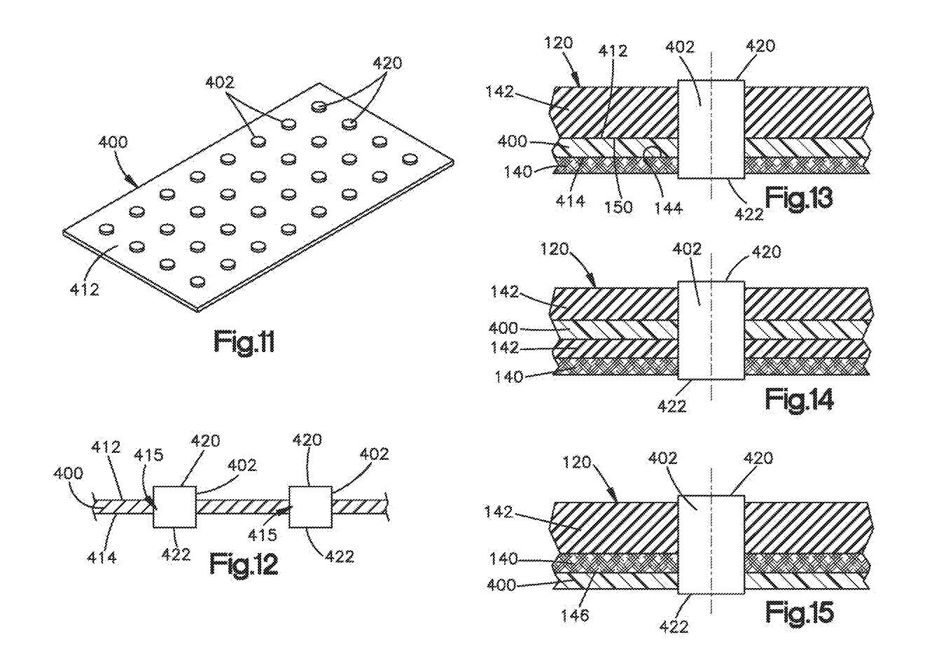

The sheet 400 can be used to support the contact elements 402 in a conductive human interface as described above. For example, the sheet 400 can be installed in a liner like the liner 120 of FIGS. 1 and 2. As shown in FIG. 13, the sheet 400 is installed as a layer between the fabric layer 140 and the soft coating 142. One side surface 414 of the sheet 400 overlies the interior surface 144 of the fabric layer 140. The exterior surface of 150 of the soft coating 144 overlies the opposite side surface 412 of the sheet 400. As shown in FIG. 14, the sheet 400 can be embedded within the soft coating 142. As shown in FIG. 15, the sheet 400 can be mounted on the exterior surface 146 of the fabric layer 140.

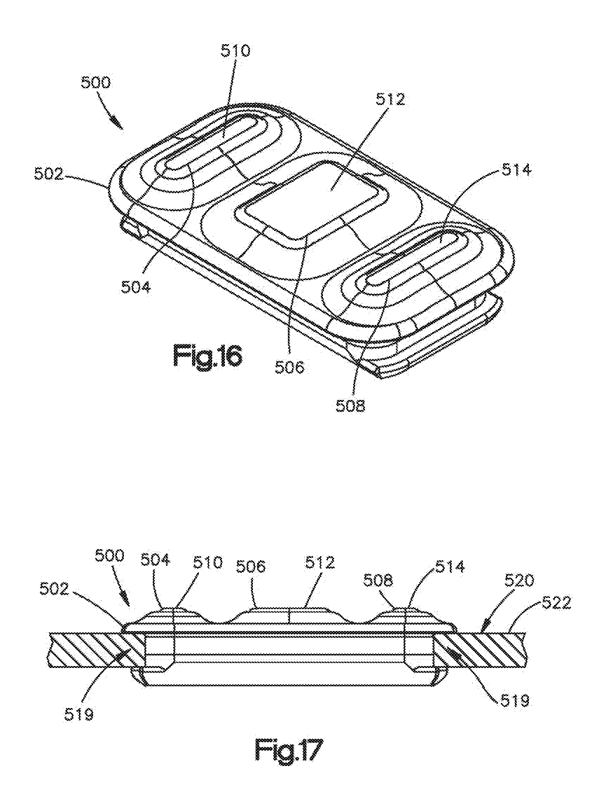

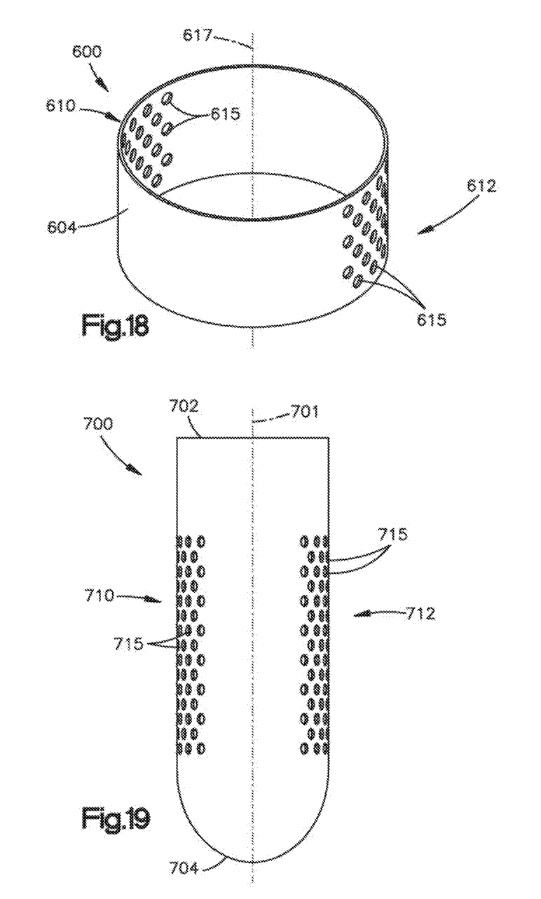

An electrode 500 device for use with the support structure 400 is shown in FIGS. 16 and 17. The electrode device 500 includes a body 502 of electrically insulating material, with inserts 504, 506 and 508 formed of electrically conductive material such as titanium. The inserts 504, 506 and 508 serve as distinct electrodes with planar electrical contact surfaces 510, 512, and 514, respectively. The contact surfaces 510, 512, and 514 are sized, shaped, and spaced apart from the each other to make signal-transmitting contact with a respective group of the contact elements 402 shown in FIG. 11, and to do so without making signal-transmitting contact with any contact element 402 in common with another of the contact surfaces 510, 512 or 514. Grooves 519 (FIG. 17) at opposite ends of the body 502 are provided to secure the electrode device 500 in an installed position embedded in a wall of a socket 520, with the contact surfaces 510, 512 and 514 exposed at the interior surface 522 of the socket 520, as described above with reference to the embodiment of FIG. 3.

Another embodiment of a support structure 600 is shown in FIG. 18. This support structure 600 is configured as an electrically insulating sheet 604 with multiple arrays 610 and 612 of apertures 615 for receiving electrical contact elements like the contact elements 402 of FIG. 11. However, the sheet 604 of FIG. 18 is continuous circumferentially about an axis 617. The sheet 604 is thus configured to be donned as a band encircling a residual limb. The arrays 610 and 612 of apertures 615 are located on the sheet 604 at circumferentially spaced-apart locations that are predetermined with reference to the neuromuscular structure of the residual limb.

In the additional embodiment of FIG. 19, a support structure 700 also is configured to be donned over a residual limb. This embodiment of a support structure 700 has a shape similar to that of the liner 120 of FIG. 1 or the socket 200 of FIG. 3. Accordingly, the support structure 700 has a longitudinal axis 701, an open proximal end 702, and a closed distal end 704. Multiple arrays 710 and 712 of apertures 715 are provided receiving electrical contact elements like the contact elements 402 of FIG. 11. The arrays 710 and 712 of apertures 715 have circumferentially spaced-apart locations that are predetermined with reference to the neuromuscular structure of the residual limb.

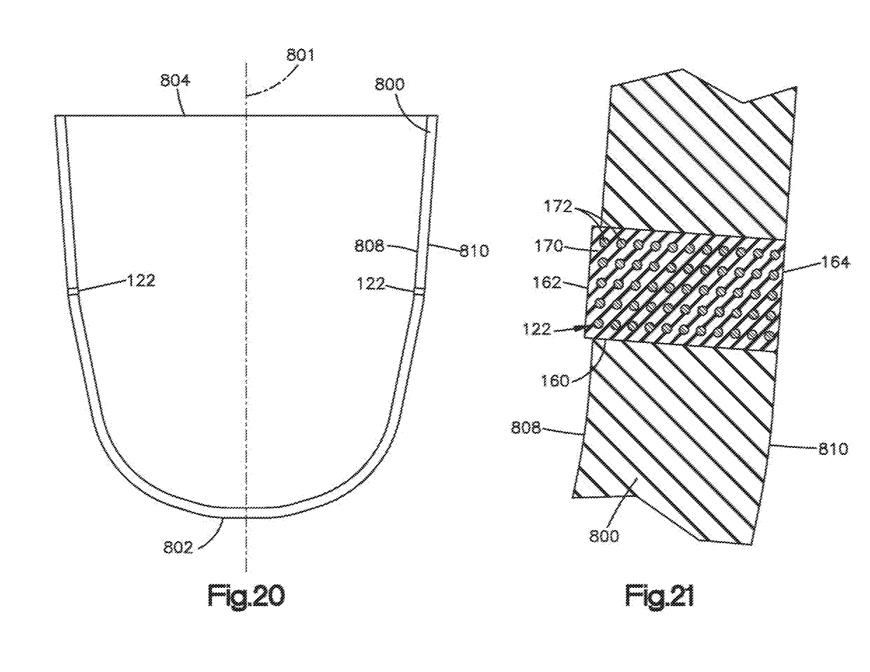

The additional embodiment of FIGS. 20 and 21 includes an electrically insulating body of elastomeric material 800. Like the soft coating 142 of FIGS. 1 and 2, the body 800 has a generally conical shape with a longitudinal axis 801, a closed distal end 802, and an open proximal end 804 for receiving a residual limb. The body 800 also has an interior surface 808 exposed for overlying contact with the user's skin at the residual limb. However, unlike the soft coating 142, the body 800 is not covered by a fabric layer. Instead, the body 800 in this embodiment is intended for the socket 200 of FIG. 3 to be received directly over the exterior surface 810 of the body 800 without the use of an intervening fabric layer.

As further shown in FIGS. 20 and 21, the body 800 serves as a support structure for electrical contact elements such as, for example, the contact elements 122 described above. Each contact element 122 reaches through the body 800 such that the interior electrical contact surface 162 is exposed at the interior surface 808 of the body 800, and the exterior electrical contact surface 164 is exposed at the exterior surface 810 of the body 800. The interior electrical contact surfaces 162 are thus exposed for overlying contact with the skin of the user at the residual limb. The exterior electrical contact surfaces 164 are thus exposed for contact with the electrodes 230 in the socket 200.

This written description sets for the best mode of carrying out the invention, and describes the invention so as to enable a person of ordinary skill in the art to make and use the invention, by presenting examples of the elements recited in the claims. The detailed descriptions of those elements do not impose limitations that are not recited in the claims, either literally or under the doctrine of equivalents.

1. An apparatus for conducting electrical signals at the skin of a user, the apparatus comprising:
a fabric layer having an interior surface and an exterior surface;
an electrically insulating elastomeric coating having an exterior surface overlying the interior surface of the fabric layer, and further having an interior surface exposed for contact with the skin of the user; and
a body of polymeric material with an electrically conductive additive dispersed in the polymeric material, wherein the body of polymeric material reaches through the fabric layer and the coating, has an interior electrical contact surface exposed at the interior surface of the coating, and further has an exterior electrical contact surface exposed at the exterior surface of the fabric layer.

2. An apparatus as defined in claim 1, further comprising a separate component including an electrode, wherein the separate component is configured to interconnect the electrode with an assistive device that is operable in electrical communication with the electrode, the separate component is further configured to be placed over the exterior surface of the fabric layer in a predetermined operative position relative to the fabric layer, and the electrode is arranged to contact the exterior electrical contact surface on the body of polymeric material when the separate component is in the predetermined operative position.

3. An apparatus as defined in claim 2, wherein the electrode is a metal electrode.

4. An apparatus as defined in claim 2, wherein the fabric layer and the coating define a prosthetic liner configured for donning over a residual limb.

5. An apparatus as defined in claim 4, wherein the separate component is a prosthetic socket.

6. An apparatus for conducting electrical signals at the skin of a user, the apparatus comprising:
a fabric layer having an interior surface and an exterior surface;
an electrically insulating coating having an exterior surface overlying the interior surface of the fabric layer, and further having an interior surface exposed for contact with the skin of the user; and
a plurality of electrical contact elements, each of which comprises a body of polymeric material with an electrically conductive additive dispersed in the polymeric material;
wherein each body of polymeric material reaches through the fabric layer and the coating, has an interior electrical contact surface exposed at the interior surface of the coating, and further has an exterior electrical contact surface exposed at the exterior surface of the fabric layer; and
a separate component including an electrode;
wherein the separate component is configured to interconnect the electrode with an assistive device that is operable in electrical communication with the electrode, the separate component is further configured to be placed over the exterior surface of the fabric layer in a predetermined position relative to the fabric layer, and the electrode is configured to contact a plurality of the exterior electrical contact surfaces on the contact elements when the separate component is in the predetermined position.

7. An apparatus as defined in claim 6, wherein the separate component includes multiple electrodes, each of which is configured to contact a plurality of the exterior electrical contact surfaces on the contact elements when the separate component is in the predetermined position, and the electrodes are arranged such that no two electrodes contact a common exterior electrical contact surface when the separate component is in the predetermined position.

8. An apparatus as defined in claim 6, wherein the fabric layer and the coating define a prosthetic liner configured for donning over a residual limb.

9. An apparatus as defined in claim 8, wherein the separate component is a prosthetic socket.

10. An apparatus as defined in claim 6, further comprising an electrically insulating support structure supporting the contact elements in a fixed array separately from the fabric layer and the coating.

11. An apparatus as defined in claim 10, wherein the support structure is received between the fabric layer and the coating.

12. An apparatus as defined in claim 10, wherein the support structure is embedded within the coating.

13. An apparatus as defined in claim 10, wherein the support structure is mounted on the exterior surface of the fabric layer.

14. An apparatus as defined in claim 10, wherein the support structure comprises a sheet having an interior side surface, an exterior side surface, and apertures reaching through the sheet between the interior and exterior side surfaces, and wherein the contact elements are received within the apertures.

15. An apparatus as defined in claim 14, wherein the contact elements are press-fitted within the apertures.

16. An apparatus as defined in claim 14, wherein the contact elements project inwardly from the interior side surface of the sheet and outwardly from the exterior side surface of the sheet.

17. An apparatus for conducting electrical signals at the skin of a user, the apparatus comprising:
a fabric layer having an interior surface and an exterior surface;
an electrically elastomeric coating having an exterior surface overlying the interior surface of the fabric layer, and further having an interior surface exposed for contact with the skin of the user; and
a plurality of electrical contact elements, each of which comprises a body of polymeric material with an electrically conductive additive dispersed in the polymeric material;
wherein each body of polymeric material reaches through the fabric layer and the coating, has an interior electrical contact surface exposed at the interior surface of the coating, and further has an exterior electrical contact surface exposed at the exterior surface of the fabric layer; and
an electrically insulating support structure supporting the contact elements in a fixed array separately from the fabric layer and the coating.

18. An apparatus as defined in claim 17, wherein the support structure is received between the fabric layer and the coating.

19. An apparatus as defined in claim 17, wherein the support structure is embedded within the coating.

20. An apparatus as defined in claim 17, wherein the support structure is mounted on the exterior surface of the fabric layer.

21. An apparatus as defined in claim 20, wherein the support structure comprises a sheet having an interior side surface, an exterior side surface, and apertures reaching through the sheet between the interior and exterior side surfaces, and wherein the contact elements are received within the apertures.

22. An apparatus as defined in claim 21, wherein the contact elements are press-fitted within the apertures.

23. An apparatus as defined in claim 21, wherein the contact elements project inwardly from the interior side surface of the sheet and outwardly from the exterior side surface of the sheet.

24. An apparatus as defined in claim 17, wherein the support structure is configured to be donned as a band encircling a residual limb.

25. An apparatus for conducting electrical signals at the skin of a user, the apparatus comprising:
a plurality of electrical contact elements, each of which comprises a body of polymeric material with an electrically conductive additive dispersed in the polymeric material; and
an electrically insulating support structure supporting the contact elements in a fixed array and having an interior surface and an exterior surface;
wherein each body of polymeric material reaches through the support structure, has an interior electrical contact surface exposed at the interior surface of the support structure, and further has an exterior electrical contact surface exposed at the exterior surface of the support structure, whereby the exterior electrical contact surface is exposed for making contact with an electrode on a separate component.

26. An apparatus as defined in claim 25 further comprising a separate component including an electrode, wherein the separate component is configured to interconnect the electrode with an assistive device that is operable in electrical communication with the electrode, the separate component is further configured to be placed in a predetermined position relative to the support structure, and the electrode is configured to contact a plurality of the exterior electrical contact surfaces on the electrical contact elements when the separate component is in the predetermined position.

27. An apparatus as defined in claim 25, wherein the support structure is configured to be donned as a band encircling a residual limb.

28. An apparatus as defined in claim 25, wherein the support structure has a generally conical shape with a closed distal end and an open proximal end for receiving a residual limb.

29. An apparatus as defined in claim 25, wherein each body of polymeric material has opposite ends and a length section oriented longitudinally between the opposite ends, the interior and exterior electrical contact surfaces are located at the opposite ends, and the length section of the body is bonded to the support structure.

30. An apparatus for conducting electrical signals at the skin of a user, the apparatus comprising:
an electrically insulating body having an interior surface and an exterior surface, wherein the interior surface is configured for contact with the skin of the user;
a plurality of electrical contact elements, each of which comprises a body of polymeric material with an electrically conductive additive dispersed in the polymeric material; and
an electrically insulating support structure supporting the contact elements in a fixed array and embedded within the insulating body;
wherein each body of polymeric material reaches through the insulating body, has an interior electrical contact surface exposed at the interior surface of the insulating body, and further has an exterior electrical contact surface exposed at the exterior surface of the insulating body.

31. An apparatus as defined in claim 30, further comprising a separate component including an electrode, wherein the separate component is configured to interconnect the electrode with an assistive device that is operable in electrical communication with the electrode, the separate component is further configured to be placed over the insulating body in a predetermined position relative to the insulating body, and the electrode is configured to contact a plurality of the exterior electrical contact surfaces on the contact elements when the separate component is in the predetermined position.

32. An apparatus as defined in claim 30, wherein the electrically insulating body is configured for donning over a residual limb.

33. An apparatus as defined in claim 31, wherein the support structure has a generally conical shape with a closed distal end and an open proximal end for receiving a residual limb.

34. An apparatus as defined in claim 30, wherein the support structure comprises a sheet having an interior side surface, an exterior side surface, and apertures reaching through the sheet between the interior and exterior side surfaces, and wherein the contact elements are received within the apertures.

35. An apparatus as defined in claim 34, wherein the contact elements are press-fitted within the apertures.

36. An apparatus as defined in claim 34, wherein the contact elements project inwardly from the interior side surface of the sheet and outwardly from the exterior side surface of the sheet.
